(19) United States
(12) Patent Application Publication
(10) Pub. No.: US 2015/0290204 A1
(43) Pub. Date: Oct. 15, 2015

(54) COMBINATION THERAPY

(71) Applicant: NOVARTIS AG, Basel (CH)

(72) Inventors: Qing Sheng, Sharon, MA (US); Hui-Qin Wang, Lexington, MA (US); Fang Li, Arlington, MA (US); Jinsheng Liang, Shrewsbury, MA (US); Zhu Alexander Cao, Acton, MA (US); John Monahan, Walpole, MA (US); Richard Versace, Wanaque, NJ (US)

(73) Assignee: Novartis AG, Basel (CH)

(21) Appl. No.: 14/440,915

(22) PCT Filed: Nov. 6, 2013
(86) PCT No.: PCT/US2013/068691

§ 371 Date: May 6, 2015

Related U.S. Application Data
(60) Provisional application No. 61/723,474, filed on Nov. 7, 2012.
(60) Provisional application No. 61/790,796, filed on Mar. 15, 2013.

(51) Int. Cl. A61K 31/5377; A61K 31/506

(52) U.S. Cl. CPC A61K 31/5377; A61K 31/506

(57) ABSTRACT

A pharmaceutical combination comprising (a) a compound of formula (I),

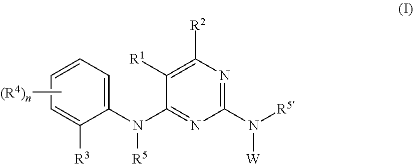

or pharmaceutically acceptable salts thereof; and (b) one or more at least one compound targeting, decreasing or inhibiting the intrinsic ATPase activity of Hsp90 and/or degrading, targeting, decreasing or inhibiting the Hsp90 client proteins via the ubiquitin proteosome pathway; the uses of such combination in the treatment or prevention of proliferative diseases; and methods of treating a subject suffering from a proliferative disease; and methods of treating a subject suffering from a proliferative disease comprising administering a therapeutically effective amount of such combination.

### FIELD OF THE INVENTION

### Background of the Invention

A compound having Formula (I):

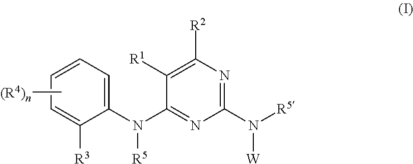

or pharmaceutically acceptable salts thereof; wherein

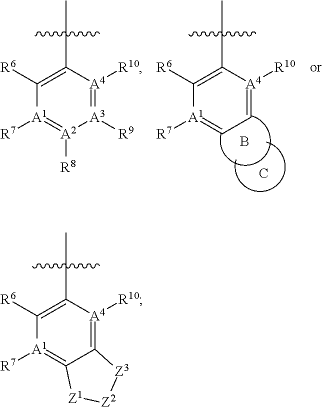

W is

A¹ and A⁴ are independently C or N;

each A² and A³ is C, or one of A² and A³ is N when R⁶ and R⁷ form a ring;

B and C are independently an optionally substituted 5-7 membered carbocyclic ring, aryl, heteroaryl or heterocyclic ring containing N, O or S;

Z¹, Z² and Z³ are independently NR¹¹, C═O, CR—OR, (CR₂)₁₋₂ or ═C—R¹²;

R¹ and R² are independently halo, OR¹², NR(R¹²), SR¹², or an optionally substituted C₁₋₆ alkyl, C₂₋₆ alkenyl or C₂₋₆ alkynyl; or one of R¹ and R² is H;

R³ is (CR₂)₀₋₂SO₂R¹², (CR₂)₀₋₂SO₂NRR¹², (CR₂)₀₋₂CO₁₋₂R¹², (CR₂)₀₋₂CONRR¹² or cyano;

R⁴, R⁶, R⁷ and R¹⁰ are independently an optionally substituted C₁₋₆ alkyl, C₂₋₆ alkenyl or C₂₋₆ alkynyl; OR¹², NR(R¹²), halo, nitro, SO₂R¹², (CR₂)ₚR¹³ or X; or R⁴, R⁷ and R¹⁰ are independently H;

R, R⁵ and R^{5′} are independently H or C₁₋₆ alkyl;

R⁸ and R⁹ are independently C₁₋₆ alkyl, C₂₋₆ alkenyl, C₂₋₆ alkynyl, halo or X, or one of R⁸ and R⁹ is H when R¹ and R² form a ring; and provided one of R⁸ and R⁹ is X;

alternatively, R¹ and R², or R⁶ and R⁷, R⁷ and R⁸, or R⁹ and R¹⁰, when attached to a carbon atom may form an optionally substituted 5-7 membered monocyclic or fused carbocyclic ring, aryl, or heteroaryl or heterocyclic ring comprising N, O and/or S; or R⁷, R⁸, R⁹ and R¹⁰ are absent when attached to N;

R¹¹ is H, C₁₋₆ alkyl, C₂₋₆ alkenyl, (CR₂)ₚCO₁₋₂R, (CR₂)ₚOR, (CR₂)ₚR¹³, (CR₂)ₚNRR¹², (CR₂)ₚCONRR¹² or (CR₂)ₚSO₁₋₂R¹²;

R¹² and R¹³ are independently an optionally substituted 3-7 membered saturated or partially unsaturated carbocyclic ring, or a 5-7 membered heterocyclic ring comprising N, O and/or S; aryl or heteroaryl; or R¹² is H, C₁₋₆ alkyl;

X is (CR₂)_{q}Y, cyano, CO₁₋₂R¹², CONR(R¹²), CONR(CR₂)ₚNR(R¹²), CONR(CR₂)ₚOR¹², CONR(CR₂)ₚSR¹², CONR(CR₂)ₚS(O)₁₋₂R¹² or (CR₂)₁₋₆NR(CR₂)ₚOR¹²;

Y is an optionally substituted 3-12 membered carbocyclic ring, a 5-12 membered aryl, or a 5-12 membered heteroaryl or heterocyclic ring comprising N, O and/or S and attached to A² or A³ or both via a carbon atom of said heteroaryl or heterocyclic ring when q in (CR₂)_{q}Y is 0; and n, p and q are independently 0-4;

were originally described in WO 2008/073687 A1.

Further, heat shock protein 90 (Hsp90) is recognized as an anti-cancer target. Hsp90 is a highly abundant and essential protein which functions as a molecular chaperone to ensure the conformational stability, shape and function of client proteins. The Hsp90 family of chaperones is comprised of four members: Hsp90a and Hsp9033 both located in the cytosol, GRP94 in the endoplasmic reticulum, and TRAP1 in the mitochondria. Hsp90 is an abundant cellular chaperone constituting about 1%-2% of total protein.

Among the stress proteins, Hsp90 is unique because it is not required for the biogenesis of most polypeptides. Hsp90 forms complexes with oncogenic proteins, called “client proteins”, which are conformationally labile signal transducers playing a critical role in growth control, cell survival and tissue development. Such binding prevents the degradation of these client proteins. A subset of Hsp90 client proteins, such as Raf, AKT, phospho-AKT, CDK4 and the EGFR family including ErbB2, are oncogenic signaling molecules critically involved in cell growth, differentiation and apoptosis, which are all processes important in cancer cells. Inhibition of the intrinsic ATPase activity of Hsp90 disrupts the Hsp90-client protein interaction resulting in their degradation via the ubiquitin proteasome pathway.

Hsp90 chaperones, which possess a conserved ATP-binding site at their N-terminal domain belong to a small ATPase sub-family known as the DNA Gyrase, Hsp90, Histidine Kinase and MutL (GHKL) sub-family. The chaperoning (folding) activity of Hsp90 depends on its ATPase activity which is weak for the isolated enzyme. However, it has been shown that the ATPase activity of Hsp90 is enhanced upon its association with proteins known as co-chaperones. Therefore, in vivo, Hsp90 proteins work as subunits of large, dynamic protein complexes. Hsp90 is essential for eukaryotic cell survival and is overexpressed in many tumors.

In spite of numerous treatment options for proliferative disease patients, there remains a need for effective and safe therapeutic agents and a need for their preferential use in combination therapy. Surprisingly, it has been found that the compounds of formula (I), which have been described in WO2008/073687, provoke strong anti-proliferative activity and an in vivo antitumor response in combination with Hsp90 inhibitors. An additional benefit of Hsp90 inhibition may arise from its effect on other signaling components within the PI3K/Akt/mTOR pathway, as for example on AKT and pAKT, and its broad effects on many client proteins.

### SUMMARY OF THE INVENTION

The present invention relates to a pharmaceutical combination comprising (a) a compound of formula (I),

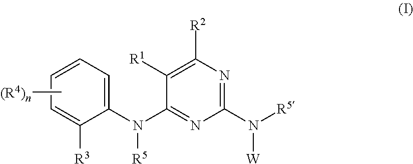

or pharmaceutically acceptable salts thereof; wherein

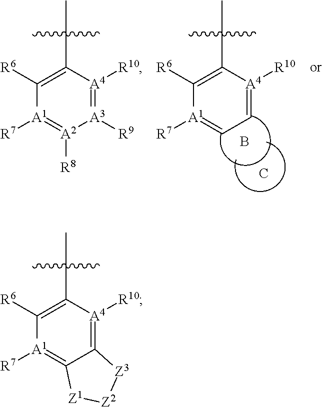

W is

A¹ and A⁴ are independently C or N;

each A² and A³ is C, or one of A² and A³ is N when R⁶ and R⁷ form a ring;

B and C are independently an optionally substituted 5-7 membered carbocyclic ring, aryl, heteroaryl or heterocyclic ring containing N, O or S;

Z¹, Z² and Z³ are independently NR¹¹, C═O, CR—OR, (CR₂)₁₋₂ or ═C—R¹²;

R¹ and R² are independently halo, OR¹², NR(R¹²), SR¹², or an optionally substituted C₁₋₆ alkyl, C₂₋₆ alkenyl or C₂₋₆ alkynyl; or one of R¹ and R² is H;

R³ is (CR₂)₀₋₂SO₂R¹², (CR₂)₀₋₂SO₂NRR¹², (CR₂)₀₋₂CO₁₋₂R¹², (CR₂)₀₋₂CONRR¹² or cyano;

R⁴, R⁶, R⁷ and R¹⁰ are independently an optionally substituted C₁₋₆ alkyl, C₂₋₆ alkenyl or C₂₋₆ alkynyl; OR¹², NR(R¹²), halo, nitro, SO₂R¹², (CR₂)ₚR¹³ or X; or R⁴, R⁷ and R¹⁰ are independently H;

R, R⁵ and R^{5′} are independently H or C₁₋₆ alkyl;

R⁸ and R⁹ are independently C₁₋₆ alkyl, C₂₋₆ alkenyl, C₂₋₆ alkynyl, halo or X, or one of R⁸ and R⁹ is H when R¹ and R² form a ring; and provided one of R⁸ and R⁹ is X;

alternatively, R¹ and R², or R⁶ and R⁷, R⁷ and R⁸, or R⁹ and R¹⁰, when attached to a carbon atom may form an optionally substituted 5-7 membered monocyclic or fused carbocyclic ring, aryl, or heteroaryl or heterocyclic ring comprising N, O and/or S; or R⁷, R⁸, R⁹ and R¹⁰ are absent when attached to N;

R¹¹ is H, C₁₋₆ alkyl, C₂₋₆ alkenyl, (CR₂)ₚCO₁₋₂R, (OR₂)ₚOR, (OR₂)ₚR¹³, (CR₂)ₚNRR¹², (CR₂)ₚCONRR¹² or (CR₂)ₚSO₁₋₂R¹²;

R¹² and R¹³ are independently an optionally substituted 3-7 membered saturated or partially unsaturated carbocyclic ring, or a 5-7 membered heterocyclic ring comprising N, O and/or S; aryl or heteroaryl; or R¹² is H, C₁₋₆ alkyl;

X is (CR₂)_{q}Y, cyano, CO₁₋₂R¹², CONR(R¹²), CONR(CR₂)ₚNR(R¹²), CONR(CR₂)ₚOR¹², CONR(CR₂)ₚSR¹², CONR(CR₂)ₚS(O)₁₋₂R¹² or (CR₂)₁₋₆NR(CR₂)ₚOR¹²;

Y is an optionally substituted 3-12 membered carbocyclic ring, a 5-12 membered aryl, or a 5-12 membered heteroaryl or heterocyclic ring comprising N, O and/or S and attached to A² or A³ or both via a carbon atom of said heteroaryl or heterocyclic ring when q in (CR₂)_{q}Y is 0; and

(b) at least one compound targeting, decreasing or inhibiting the intrinsic ATPase activity of Hsp90 and/or degrading, targeting, decreasing or inhibiting the Hsp90 client proteins via the ubiquitin proteosome pathway. Such compounds will be referred to as “Heat shock protein 90 inhibitors” or “Hsp90 inhibitors. Examples of Hsp90 inhibitors suitable for use in the present invention include, but are not limited to, the geldanamycin derivative, Tanespimycin (17-allylamino-17-demethoxygeldanamycin)(also known as KOS-953 and 17-AAG); Radicicol; 6-Chloro-9-(4-methoxy-3,5-dimethylpyridin-2-ylmethyl)-9H-purin-2-amine methanesulfonate (also known as CNF2024); IPI504; SNX5422; 5-(2,4-Dihydroxy-5-isopropyl-phenyl)-4-(4-morpholin-4-ylmethyl-phenyl)-isoxazole-3-carboxylic acid ethylamide (AUY922); and (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990).

In the above Formula (1), R¹ may be halo or C₁₋₆ alkyl; R² is H or NH₂; or R¹ and R² together form an optionally substituted 5-6 membered aryl, or heteroaryl or heterocyclic ring comprising 1-3 nitrogen atoms, In other examples, R³ in Formula (1) may be SO₂R¹², SO₂NH₂, SO₂NRR¹², CO₂NH₂, CONRR¹², CO₁₋₂R¹², or cyano; and R¹² is C₁₋₆ alkyl, an optionally substituted C₃₋₇ cycloalkyl, C₃₋₇ cycloalkenyl, pyrrolidinyl, piperazinyl, piperidinyl, morpholinyl or azetidinyl In yet other examples, R⁵, R^{5′}, R⁷ and R¹⁰ in Formula (1) are independently H, and n is 0, In other examples, R⁶ in Formula (1) may be halo or OR¹², and R¹² is C₁₋₆ alkyl.

In a preferred embodiment, the compound of Formula (1) is

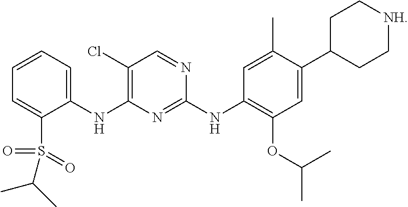 [SMILES: Cc1cc(Nc2ncc(Cl)c(Nc3ccccc3S(=O)(=O)C(C)C)n2)c(OC(C)C)cc1C1CCNCC1]

The present invention further relates to a pharmaceutical composition comprising a compound of formula (I) or a pharmaceutically acceptable salt thereof and at least one Hsp90 inhibitor or a pharmaceutically acceptable salt thereof. In one embodiment, this pharmaceutical composition of the present invention is for use in the treatment of a proliferative disease.

The present invention further relates to the use of a pharmaceutical combination comprising a compound of formula (I) or a pharmaceutically acceptable salt thereof and at least one Hsp90 inhibitor or a pharmaceutically acceptable salt thereof, for the preparation of a medicament for the treatment of a proliferative disease.

The present invention further relates to a method for treating a proliferative disease in a subject in need thereof, comprising administering to said subject a therapeutically effective amount of a compound of formula (I) or a pharmaceutically acceptable salt thereof, and at least one Hsp90 inhibitor or a pharmaceutically acceptable salt thereof. In accordance with the present invention, the compound of formula (I) and the Hsp90 inhibitor may be administered either as a single pharmaceutical composition, as separate compositions, or sequentially.

The present invention further relates to a kit comprising a compound of formula (I) according to claim 1 or a pharmaceutically acceptable salt thereof, and at least one Hsp90 inhibitor or a pharmaceutically acceptable salt thereof.

In one embodiment of the present invention, the compound of formula (I) is selected from 5-chloro-N2-(2-isopropoxy-5-methyl-4-(piperidin-4-yl)phenyl)-N4-[2-(propane-2-sulfonyl)-phenyl]-pyrimidine-2,4-diamine (Compound A) having the following structure

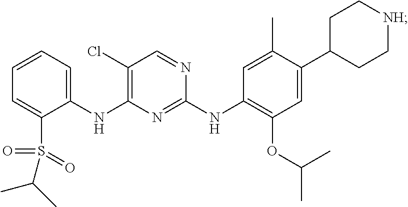 [SMILES: Cc1cc(Nc2ncc(Cl)c(Nc3ccccc3S(=O)(=O)C(C)C)n2)c(OC(C)C)cc1C1CCNCC1]

or pharmaceutically acceptable salts thereof.

In one embodiment of the present invention, the HSP inhibitor is 5-(2,4-Dihydroxy-5-isopropyl-phenyl)-4-(4-morpholin-4-ylmethyl-phenyl)-isoxazole-3-carboxylic acid ethylamide (AUY922).

In one embodiment of the present invention, the compound of formula (I) is 5-chloro-N2-(2-isopropoxy-5-methyl-4-(piperidin-4-yl)phenyl)-N4-[2-(propane-2-sulfonyl)-phenyl]-pyrimidine-2,4-diamine (Compound A) and the HSP inhibitor is 5-(2,4-Dihydroxy-5-isopropyl-phenyl)-4-(4-morpholin-4-ylmethyl-phenyl)-isoxazole-3-carboxylic acid ethylamide (AUY922).

### DESCRIPTION OF THE FIGURES

FIG. 1 shows the antitumor activity of AUY922 50 mg/kg, 5-chloro-N2-(2-isopropoxy-5-methyl-4-(piperidin-4-yl)phenyl)-N4-[2-(propane-2-sulfonyl)-phenyl]-pyrimidine-2,4-diamine (Compound A) 10 mg/kg, or combination of AUY922 50 mg/kg and Compound A 10 mg/kg in mice bearing HLUX-1787 lung primary tumor xenografts which harbor an EML4-ALK variant 2 translocation (TRP-0318).

FIG. 2 shows the percent change in body weight of AUY922 50 mg/kg, Compound A 10 mg/kg, or combination of AUY922 50 mg/kg and Compound A 10 mg/kg in mice bearing HLUX-1787 lung primary tumor xenografts which harbor an EML4-ALK variant 2 translocation (TRP-0318).

For the in vivo testing in FIGS. 1 and 2, female nude (nu/nu) harlan mice bearing HLUX-1787 lung primary tumor xenografts were treated with AUY922, Compound A, a combination of AUY922 and Compound A, or vehicle at the indicated doses and schedules. Treatments started 24 days post tumor cells implantation and lasted 20 consecutive days. Statistics on change in tumor volumes and were performed with a one-way ANOVA, post hoc Tukey (* p<0.05 vs. vehicle controls).

FIG. 3 shows the antitumor activity of AUY922 50 mg/kg, Compound A 10 mg/kg, or combination of AUY922 50 mg/kg and Compound A 10 mg/kg in mice bearing HLUX-1787 lung primary tumor xenografts which harbor an EML4-ALK variant 2 translocation (TRP-0335).

FIG. 4 shows the percent change in body weight of AUY922 50 mg/kg, Compound A 10 mg/kg, or combination of AUY922 50 mg/kg and Compound A 10 mg/kg in mice bearing HLUX-1787 lung primary tumor xenografts which harbor an EML4-ALK variant 2 translocation (TRP-0318).

For the in vivo testing in FIGS. 3 and 4, female nude (nu/nu) harlan mice bearing HLUX-1787 lung primary tumor xenografts were treated with AUY922, Compound A, a combination of AUY922 and Compound A, or vehicle at the indicated doses and schedules. Treatments started 27 days post tumor cells implantation and lasted 13 consecutive days. Statistics on change in tumor volumes and were performed with a one-way ANOVA, post hoc Tukey (* p<0.05 vs. vehicle controls).

FIG. 5 shows the mean body weight of vehicle, Compound A 25 mg/kg, Compound A 50 mg/kg, Compound A 100 mg/kg, AUY922 50 mg/kg, and combination AUY922 50 mg/kg and Compound A 25 mg/kg treated groups in mice bearing the subcutaneous primary human lung cancer LUF1656 (treatment phase, n=8) by day 21.

FIG. 6 shows the mean body weight of vehicle, Compound A 25 mg/kg, Compound A 50 mg/kg, Compound A 100 mg/kg, AUY922 50 mg/kg, and combination AUY922 50 mg/kg and Compound A 25 mg/kg treated groups in mice bearing the subcutaneous primary human lung cancer LUF1656 (re-growth phase, n=4) from day 22 to day 34.

FIG. 7 shows the antitumor activity of Compound A 25 mg/kg, Compound A 50 mg/kg, Compound A 100 mg/kg, AUY922 50 mg/kg, and combination AUY922 50 mg/kg and Compound A 25 mg/kg treated groups in mice bearing the subcutaneous primary human lung cancer LUF1656 (treatment phase, n=8) by day 21.

FIG. 8 shows the antitumor activity of Compound A 25 mg/kg, Compound A 50 mg/kg, Compound A 100 mg/kg, AUY922 50 mg/kg, and combination AUY922 50 mg/kg and Compound A 25 mg/kg treated groups in mice bearing the subcutaneous primary human lung cancer LUF1656 (re-growth phase, n=4) from day 22 to day 34.

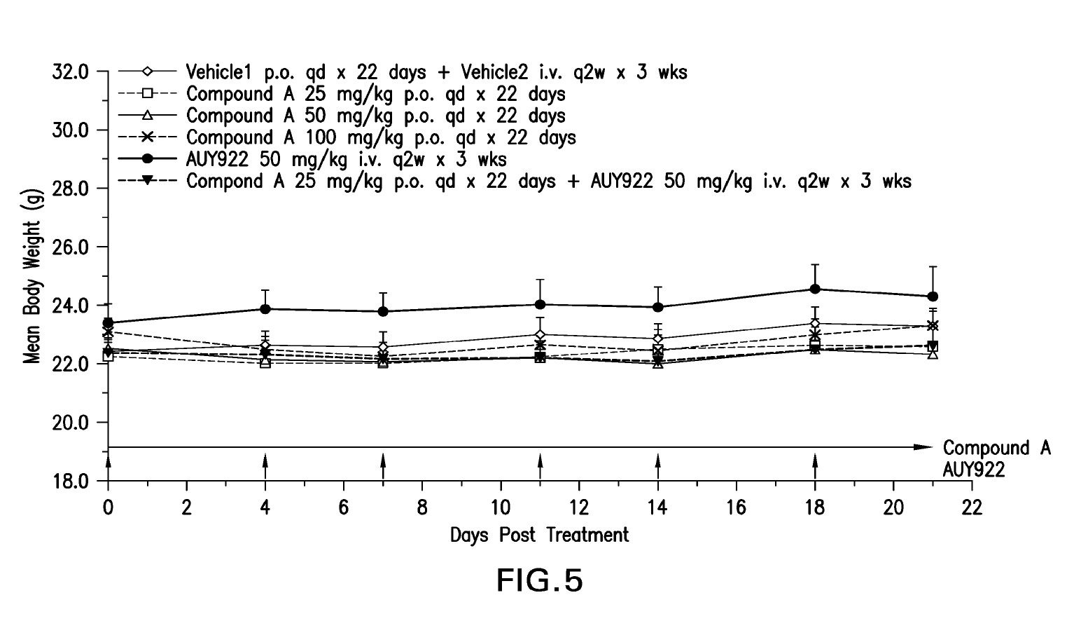

For the in vivo testing in FIGS. 5, 6, 7 and 8, female nude (nu/nu) mice bearing LUF1656 lung primary tumor xenografts were treated with AUY922, Compound A, a combination of AUY922 and Compound A, or vehicle at the indicated doses and schedules. The treatments were started when mean tumor size reached approximately 140 mm³ (range 86.8-245 mm³). Statistics on change in tumor volumes and were performed with a one-way ANOVA, post hoc Tukey (* p<0.05 vs. vehicle controls).

### DETAILED DESCRIPTION OF THE INVENTION

The following general definitions are provided to better understand the invention:

### DEFINITIONS

“Alkyl” refers to a moiety and as a structural element of other groups, for example halo-substituted-alkyl and alkoxy, and may be straight-chained or branched. An optionally substituted alkyl, alkenyl or alkynyl as used herein may be optionally halogenated (e.g., CF₃), or may have one or more carbons that is substituted or replaced with a heteroatom, such as NR, O or S (e.g., —OCH₂CH₂O—, alkylthiols, thioalkoxy, alkylamines, etc).

“Aryl” refers to a monocyclic or fused bicyclic aromatic ring containing carbon atoms. “Arylene” means a divalent radical derived from an aryl group. For example, an aryl group may be phenyl, indenyl, indanyl, naphthyl, or 1,2,3,4-tetrahydronaphthalenyl, which may be optionally substituted in the ortho, meta or para position.

“Heteroaryl” as used herein is as defined for aryl above, where one or more of the ring members is a heteroatom. Examples of heteroaryls include but are not limited to pyridyl, pyrazinyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, benzotriazolyl, tetrazolyl, pyrazolyl, thienyl, pyrrolyl, isoquinolinyl, purinyl, thiazolyl, tetrazinyl, benzothiazolyl, oxadiazolyl, benzoxadiazolyl, etc.

A “carbocyclic ring” as used herein refers to a saturated or partially unsaturated, monocyclic, fused bicyclic or bridged polycyclic ring containing carbon atoms, which may optionally be substituted, for example, with ═O. Examples of carbocyclic rings include but are not limited to cyclopropyl, cyclobutyl, cyclopentyl, cyclohexyl, cyclopropylene, cyclohexanone, etc.

A “heterocyclic ring” as used herein is as defined for a carbocyclic ring above, wherein one or more ring carbons is a heteroatom. For example, a heterocyclic ring may contain N, O, S, —N═, —S—, —S(O), —S(O)₂—, or —NR— wherein R may be hydrogen, C₁₋₄alkyl or a protecting group. Examples of heterocyclic rings include but are not limited to morpholino, pyrrolidinyl, pyrrolidinyl-2-one, piperazinyl, piperidinyl, piperidinylone, 1,4-dioxa-8-aza-spiro[4.5]dec-8-yl, 1,2,3,4-tetrahydroquinolinyl, etc. Heterocyclic rings as used herein may encompass bicyclic amines and bicyclic diamines.

“Salts” (which, what is meant by “or salts thereof” or “or a salt thereof”), can be present alone or in mixture with free compound, e.g. the compound of the formula (I), and are preferably pharmaceutically acceptable salts. Such salts of the compounds of formula (I) are formed, for example, as acid addition salts, preferably with organic or inorganic acids, from compounds of formula (I) with a basic nitrogen atom. Suitable inorganic acids are, for example, halogen acids, such as hydrochloric acid, sulfuric acid, or phosphoric acid. Suitable organic acids are, e.g., carboxylic acids or sulfonic acids, such as fumaric acid or methansulfonic acid. For isolation or purification purposes it is also possible to use pharmaceutically unacceptable salts, for example picrates or perchlorates. For therapeutic use, only pharmaceutically acceptable salts or free compounds are employed (where applicable in the form of pharmaceutical preparations), and these are therefore preferred. In view of the close relationship between the novel compounds in free form and those in the form of their salts, including those salts that can be used as intermediates, for example in the purification or identification of the novel compounds, any reference to the free compounds hereinbefore and hereinafter is to be understood as referring also to the corresponding salts, as appropriate and expedient. The salts of compounds of formula (I) are preferably pharmaceutically acceptable salts; suitable counter-ions forming pharmaceutically acceptable salts are known in the field.

“Combination” refers to either a fixed combination in one dosage unit form, or a non-fixed combination (or kit of parts) for the combined administration where a compound of the formula (I) and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The term “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “fixed combination” means that the active ingredients, e.g. a compound of formula (I) and a combination partner, are both administered to a patient simultaneously in the form of a single entity or dosage. The terms “non-fixed combination” or “kit of parts” mean that the active ingredients, e.g. a compound of formula (I) and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

“Treatment” includes prophylactic (preventive) and therapeutic treatment as well as the delay of progression of a disease or disorder. The term “prophylactic” means the prevention of the onset or recurrence of diseases involving proliferative diseases. The term “delay of progression” as used herein means administration of the combination to patients being in a pre-stage or in an early phase of the proliferative disease to be treated, in which patients for example a pre-form of the corresponding disease is diagnosed or which patients are in a condition, e.g. during a medical treatment or a condition resulting from an accident, under which it is likely that a corresponding disease will develop.

“Subject” is intended to include animals. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from a brain tumor disease. Particularly preferred, the subject is human.

“Pharmaceutical preparation” or “pharmaceutical composition” refer to a mixture or solution containing at least one therapeutic compound to be administered to a mammal, e.g., a human in order to prevent, treat or control a particular disease or condition affecting the mammal.

“Co-administer”, “co-administration” or “combined administration” or the like are meant to encompass administration of the selected therapeutic agents to a single patient, and are intended to include treatment regimens in which the agents are not necessarily administered by the same route of administration or at the same time.

“Pharmaceutically acceptable” refers to those compounds, materials, compositions and/or dosage forms, which are, within the scope of sound medical judgment, suitable for contact with the tissues of mammals, especially humans, without excessive toxicity, irritation, allergic response and other problem complications commensurate with a reasonable benefit/risk ratio.

“Therapeutically effective” preferably relates to an amount that is therapeutically or in a broader sense also prophylactically effective against the progression of a proliferative disease.

“Single pharmaceutical composition” refers to a single carrier or vehicle formulated to deliver effective amounts of both therapeutic agents to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension.

“Dose range” refers to an upper and a lower limit of an acceptable variation of the amount of agent specified. Typically, a dose of the agent in any amount within the specified range can be administered to patients undergoing treatment.

The terms “about” or “approximately” usually means within 20%, more preferably within 10%, and most preferably still within 5% of a given value or range. Alternatively, especially in biological systems, the term “about” means within about a log (i.e., an order of magnitude) preferably within a factor of two of a given value.

The present invention relates to a pharmaceutical combination comprising (a) a compound of formula (I), as defined HEREIN, or a pharmaceutically acceptable salt thereof; and (b) at least one Hsp90 inhibitor or a pharmaceutically acceptable salt thereof. Such combination may be for simultaneous, separate or sequential use for the treatment of a proliferative disease.

Suitable Hsp90 inhibitors include, but are not limited to,
(a) the geldanamycin derivative, Tanespimycin (17-allylamino-17-demethoxygeldanamycin)(also known as KOS-953 and 17-AAG), which is available from Sigma-Aldrich Co, LLC (St. Louis, Mo.), and disclosed in U.S. Pat. No. 4,261,989, dated Apr. 14, 1981, which is hereby incorporated into the present application by reference, and other geldanamycin-related compounds;
(b) Radicicol, which is available from Sigma-Aldrich Co, LLC (St. Louis, Mo.);
(c) 6-Chloro-9-(4-methoxy-3,5-dimethylpyridin-2-ylmethyl)-9H-purin-2-amine methanesulfonate (also known as CNF2024)(Conforma Therapeutics Corp.);
(d) IPI504;
(e) SNX5422;
(f) 5-(2,4-Dihydroxy-5-isopropyl-phenyl)-4-(4-morpholin-4-ylmethyl-phenyl)-isoxazole-3-carboxylic acid ethylamide (AUY922), which is disclosed in structure and with the process for its manufacture in PCT Application No. WO04/072051, published on Aug. 26, 2004, which is hereby incorporated into the present application by reference; and
(g) (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990), which is disclosed in structure and with the process for its manufacture in U.S. Patent Application Publication No. 2007-0123546, published on May 31, 2007, which is hereby incorporated into the present application by reference;

and pharmaceutically acceptable salts thereof.

Preferred Hsp90 inhibitors for the present invention are 5-(2,4-Dihydroxy-5-isopropyl-phenyl)-4-(4-morpholin-4-ylmethyl-phenyl)-isoxazole-3-carboxylic acid ethylamide (AUY922) and (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990) or pharmaceutically acceptable salts thereof.

Comprised are likewise the pharmaceutically acceptable salts thereof, the corresponding racemates, diastereoisomers, enantiomers, tautomers, as well as the corresponding crystal modifications of above disclosed compounds where present, e.g. solvates, hydrates and polymorphs, which are disclosed therein. The compounds used as active ingredients in the combinations of the present invention can be prepared and administered as described in the cited documents, respectively. Also within the scope of this invention is the combination of more than two separate active ingredients as set forth above, i.e., a pharmaceutical combination within the scope of this invention could include three active ingredients or more.

In one embodiment of the present invention, the pharmaceutical combination comprises the compound of formula (I) that is

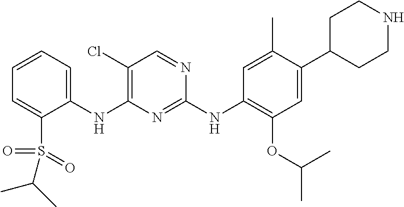 [SMILES: Cc1cc(Nc2ncc(Cl)c(Nc3ccccc3S(=O)(=O)C(C)C)n2)c(OC(C)C)cc1C1CCNCC1]

or a pharmaceutically acceptable salt thereof, and at least one Hsp90 inhibitor selected from 5-(2,4-Dihydroxy-5-isopropyl-phenyl)-4-(4-morpholin-4-ylmethyl-phenyl)-isoxazole-3-carboxylic acid ethylamide (AUY922), (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990), or pharmaceutically acceptable salts thereof.

In one embodiment of the present invention, the pharmaceutical combination comprises the compound of formula (I) that is 5-chloro-N2-(2-isopropoxy-5-methyl-4-(piperidin-4-yl)phenyl)-N4-[2-(propane-2-sulfonyl)-phenyl]-pyrimidine-2,4-diamine or pharmaceutically acceptable salts thereof, and at least one Hsp90 inhibitor 5-(2,4-Dihydroxy-5-isopropyl-phenyl)-4-(4-morpholin-4-ylmethyl-phenyl)-isoxazole-3-carboxylic acid ethylamide (AUY922) or a pharmaceutically acceptable salt thereof.

In one embodiment of the present invention, the pharmaceutical combination comprises the compound of formula (I) that is 5-chloro-N2-(2-isopropoxy-5-methyl-4-(piperidin-4-yl)phenyl)-N4-[2-(propane-2-sulfonyl)-phenyl]-pyrimidine-2,4-diamine (Compound A) having the following structure

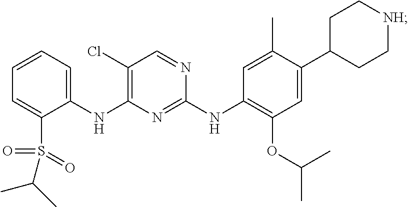 [SMILES: Cc1cc(Nc2ncc(Cl)c(Nc3ccccc3S(=O)(=O)C(C)C)n2)c(OC(C)C)cc1C1CCNCC1]

or pharmaceutically acceptable salts thereof and the HSP inhibitor is 5-(2,4-Dihydroxy-5-isopropyl-phenyl)-4-(4-morpholin-4-ylmethyl-phenyl)-isoxazole-3-carboxylic acid ethylamide (AUY922).

In a further embodiment, the compound of formula (I) is 5-chloro-N2-(2-isopropoxy-5-methyl-4-(piperidin-4-yl)phenyl)-N4-[2-(propane-2-sulfonyl)-phenyl]-pyrimidine-2,4-diamine (Compound A) and the HSP inhibitor is 5-(2,4-Dihydroxy-5-isopropyl-phenyl)-4-(4-morpholin-4-ylmethyl-phenyl)-isoxazole-3-carboxylic acid ethylamide (AUY922).

It has now been surprisingly found that the combination of a compound of formula (I), and at least one Hsp90 inhibitor possess beneficial therapeutic properties, which render it particularly useful for the treatment of proliferative diseases, particularly cancer.

In one aspect, the present invention provides a pharmaceutical combination comprising (a) a compound of formula (I), and (b) at least one Hsp90 inhibitor or a pharmaceutically acceptable salt thereof, for use in the treatment of a proliferative disease, particularly cancer.

In one aspect, the present invention provides the use of a pharmaceutical combination comprising a compound of formula (I) or a pharmaceutically acceptable salt thereof and at least one Hsp90 inhibitor or a pharmaceutically acceptable salt thereof, for the preparation of a medicament for the treatment of a proliferative disease.

In one aspect, the present invention further relates to a method for treating a proliferative disease in a subject in need thereof, comprising administering to said subject a therapeutically effective amount of a compound of formula (I) or a pharmaceutically acceptable salt thereof, and at least one Hsp90 inhibitor or a pharmaceutically acceptable salt thereof. In accordance with the present invention, the compound of formula (I) and the Hsp90 inhibitor may be administered either as a single pharmaceutical composition, as separate compositions, or sequentially.

Preferably, the present invention is useful for the treating a mammal, especially humans, suffering from a proliferative disease such as cancer.

To demonstrate that the combination of a compound of formula (I) and at least one Hsp90 inhibitor is particularly suitable for the effective treatment of proliferative diseases with good therapeutic margin and other advantages, clinical trials can be carried out in a manner known to the skilled person.

Suitable clinical studies are, e.g., open label, dose escalation studies in patients with proliferative diseases. Such studies prove in particular the synergism of the active ingredients of the combination of the invention. The beneficial effects can be determined directly through the results of these studies which are known as such to a person skilled in the art. Such studies are, in particular, suitable to compare the effects of a monotherapy using the active ingredients and a combination of the invention. Preferably, the dose of agent (a) is escalated until the Maximum Tolerated Dosage is reached, and agent (b) is administered with a fixed dose. Alternatively, the agent (a) is administered in a fixed dose and the dose of agent (b) is escalated. Each patient receives doses of the agent (a) either daily or intermittent. The efficacy of the treatment can be determined in such studies, e.g., after 12, 18 or 24 weeks by evaluation of symptom scores every 6 weeks.

The administration of a pharmaceutical combination of the invention results not only in a beneficial effect, e.g., a synergistic therapeutic effect, e.g., with regard to alleviating, delaying progression of or inhibiting the symptoms, but also in further surprising beneficial effects, e.g., fewer side effects, an improved quality of life or a decreased morbidity, compared with a monotherapy applying only one of agents (a) or agents (b) used in the combination of the invention.

A further benefit is that lower doses of the active ingredients of the combination of the invention can be used, e.g., that the dosages need not only often be smaller but are also applied less frequently, which may diminish the incidence or severity of side effects. This is in accordance with the desires and requirements of the patients to be treated.

It is one objective of this invention to provide a pharmaceutical composition comprising a quantity, which is jointly therapeutically effective at targeting or preventing proliferative diseases, of each combination partner agent (a) and (b) of the invention. In one aspect, the present invention relates to a pharmaceutical composition comprising a compound of formula (I) or a pharmaceutically acceptable salt thereof and at least one Hsp90 inhibitor or a pharmaceutically acceptable salt thereof. In one embodiment, such pharmaceutical composition of the present invention is for use in the treatment of a proliferative disease. In accordance with the present invention, agent (a) and agent (b) may be administered together in a single pharmaceutical composition, separately in one combined unit dosage form or in two separate unit dosage forms, or sequentially. The unit dosage form may also be a fixed combination.

The pharmaceutical compositions for separate administration of agent (a) and agent (b) or for the administration in a fixed combination (i.e., a single galenical composition comprising at least two combination partners (a) and (b)) according to the invention may be prepared in a manner known per se and are those suitable for enteral, such as oral or rectal, topical, and parenteral administration to subjects, including mammals (warm-blooded animals) such as humans, comprising a therapeutically effective amount of at least one pharmacologically active combination partner alone, e.g., as indicated above, or in combination with one or more pharmaceutically acceptable carriers or diluents, especially suitable for enteral or parenteral application. Suitable pharmaceutical compositions contain, e.g., from about 0.1% to about 99.9%, preferably from about 1% to about 60%, of the active ingredient(s).

Pharmaceutical compositions for the combination therapy for enteral or parenteral administration are, e.g., those in unit dosage forms, such as sugar-coated tablets, tablets, capsules or suppositories, ampoules, injectable solutions or injectable suspensions. Topical administration is e.g. to the skin or the eye, e.g. in the form of lotions, gels, ointments or creams, or in a nasal or a suppository form. If not indicated otherwise, these are prepared in a manner known per se, e.g., by means of conventional mixing, granulating, sugar-coating, dissolving or lyophilizing processes. It will be appreciated that the unit content of agent (a) or agent (b) contained in an individual dose of each dosage form need not in itself constitute an effective amount since the necessary effective amount can be reached by administration of a plurality of dosage units.

Pharmaceutical compositions may comprise one or more pharmaceutical acceptable carriers or diluents and may be manufactured in conventional manner by mixing one or both combination partners with a pharmaceutically acceptable carrier or diluent. Examples of pharmaceutically acceptable diluents include, but are not limited to, lactose, dextrose, mannitol, and/or glycerol, and/or lubricants and/or polyethylene glycol. Examples of pharmaceutically acceptable binders include, but are not limited to, magnesium aluminum silicate, starches, such as corn, wheat or rice starch, gelatin, methylcellulose, sodium carboxymethylcellulose and/or polyvinylpyrrolidone, and, if desired, pharmaceutically acceptable disintegrators include, but are not limited to, starches, agar, alginic acid or a salt thereof, such as sodium alginate, and/or effervescent mixtures, or adsorbents, dyes, flavorings and sweeteners. It is also possible to use the compounds of the present invention in the form of parenterally administrable compositions or in the form of infusion solutions. The pharmaceutical compositions may be sterilized and/or may comprise excipients, for example preservatives, stabilizers, wetting compounds and/or emulsifiers, solubilisers, salts for regulating the osmotic pressure and/or buffers.

In particular, a therapeutically effective amount of each of the combination partner of the combination of the invention may be administered simultaneously or sequentially and in any order, and the components may be administered separately or as a fixed combination. For example, the method of preventing or treating proliferative diseases according to the invention may comprise: (i) administration of the first agent (a) in free or pharmaceutically acceptable salt form; and (ii) administration of an agent (b) in free or pharmaceutically acceptable salt form, simultaneously or sequentially in any order, in jointly therapeutically effective amounts, preferably in synergistically effective amounts, e.g., in daily or intermittently dosages corresponding to the amounts described herein. The individual combination partners of the combination of the invention may be administered separately at different times during the course of therapy or concurrently in divided or single combination forms. Furthermore, the term administering also encompasses the use of a pro-drug of a combination partner that convert in vivo to the combination partner as such. The instant invention is therefore to be understood as embracing all such regimens of simultaneous or alternating treatment and the term “administering” is to be interpreted accordingly.

The effective dosage of each of combination partner agent (a) or agent (b) employed in the combination of the invention may vary depending on the particular compound or pharmaceutical composition employed, the mode of administration, the condition being treated, the severity of the condition being treated. Thus, the dosage regimen of the combination of the invention is selected in accordance with a variety of factors including type, species, age, weight, sex and medical condition of the patient; the severity of the condition to be treated; the route of administration; the renal and hepatic function of the patient; and the particular compound employed. A physician, clinician or veterinarian of ordinary skill can readily determine and prescribe the effective amount of the drug required to prevent, counter or arrest the progress of the condition. Optimal precision in achieving concentration of drug within the range that yields efficacy requires a regimen based on the kinetics of the drug's availability to target sites. This involves a consideration of the distribution, equilibrium, and elimination of a drug.

For purposes of the present invention, a therapeutically effective dose will generally be a total daily dose administered to a host in single or divided doses. The compound of formula (I) may be administered to a host in a daily dosage range of, for example, from about 0.05 to about 50 mg/kg body weight of the recipient, preferably about 0.1-25 mg/kg body weight of the recipient, more preferably from about 0.5 to 10 mg/kg body weight of the recipient. Agent (b) may be administered to a host in a daily dosage range of, for example, from about 0.001 to 1000 mg/kg body weight of the recipient, preferably from 1.0 to 100 mg/kg body weight of the recipient, and most preferably from 1.0 to 50 mg/kg body weight of the recipient. Dosage unit compositions may contain such amounts of submultiples thereof to make up the daily dose.

A further benefit is that lower doses of the active ingredients of the combination of the invention can be used, e.g., that the dosages need not only often be smaller but are also applied less frequently, or can be used in order to diminish the incidence of side effects. This is in accordance with the desires and requirements of the patients to be treated.

The combination of the compound of formula (I) and an HSP90 inhibitor can be used alone or combined with at least one other pharmaceutically active compound for use in these pathologies. These active compounds can be combined in the same pharmaceutical preparation or in the form of combined preparations “kit of parts” in the sense that the combination partners can be dosed independently or by use of different fixed combinations with distinguished amounts of the combination partners, i.e., simultaneously or at different time points. The parts of the kit of parts can then, e.g., be administered simultaneously or chronologically staggered, that is at different time points and with equal or different time intervals for any part of the kit of parts. Non-limiting examples of compounds which can be cited for use in combination with the combination of a compound of formula (I) and at least one HSP90 inhibitor are cytotoxic chemotherapy drugs, such as anastrozole, doxorubicin hydrochloride, flutamide, dexamethaxone, docetaxel, cisplatin, paclitaxel, etc. Further, the combination of a pyrimidylaminobenzamide compound and an HSP90 inhibitor could be combined with other inhibitors of signal transduction or other oncogene-targeted drugs with the expectation that significant synergy would result.

The combination of the present invention is particularly useful for the treatment of proliferative diseases. The term “proliferative disease” includes, but not restricted to, cancer, tumor, hyperplasia, restenosis, cardiac hypertrophy, immune disorder and inflammation.

Examples for a proliferative disease the can be treated with the combination of the present invention are for instance cancers, including, for example, sarcoma; lung; bronchus; prostate; breast (including sporadic breast cancers and sufferers of Cowden disease); pancreas; gastrointestinal cancer or gastric; colon; rectum; colorectal adenoma; thyroid; liver; intrahepatic bile duct; hepatocellular; adrenal gland; stomach; glioma; glioblastoma; endometrial; kidney; renal pelvis; urinary bladder; uterine corpus; uterine cervix; vagina; ovary; multiple myeloma; esophagus; a leukaemia; acute myelogenous leukemia; chronic myelogenous leukemia; lymphocytic leukemia; myeloid leukemia; brain; oral cavity and pharynx; larynx; small intestine; non-Hodgkin lymphoma; melanoma; villous colon adenoma; a neoplasia; a neoplasia of epithelial character; lymphomas; a mammary carcinoma; basal cell carcinoma; squamous cell carcinoma; actinic keratosis; a tumor of the neck or head; polycythemia vera; essential thrombocythemia; myelofibrosis with myeloid metaplasia; and Walden stroem disease.

Further examples include, polycythemia vera, essential thrombocythemia, myelofibrosis with myeloid metaplasia, asthma, COPD, ARDS, Loffler's syndrome, eosinophilic pneumonia, parasitic (in particular metazoan) infestation (including tropical eosinophilia), bronchopulmonary aspergillosis, polyarteritis nodosa (including Churg-Strauss syndrome), eosinophilic granuloma, eosinophil-related disorders affecting the airways occasioned by drug-reaction, psoriasis, contact dermatitis, atopic dermatitis, alopecia areata, erythema multiforme, dermatitis herpetiformis, scleroderma, vitiligo, hypersensitivity angiitis, urticaria, bullous pemphigoid, lupus erythematosus, pemphisus, epidermolysis bullosa acquisita, autoimmune haematogical disorders (e.g. haemolytic anaemia, aplastic anaemia, pure red cell anaemia and idiopathic thrombocytopenia), systemic lupus erythematosus, polychondritis, scleroderma, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), interstitial lung fibrosis, psoriatic arthritis, glomerulonephritis, cardiovascular diseases, atherosclerosis, hypertension, deep venous thrombosis, stroke, myocardial infarction, unstable angina, thromboembolism, pulmonary embolism, thrombolytic diseases, acute arterial ischemia, peripheral thrombotic occlusions, and coronary artery disease, reperfusion injuries, retinopathy, such as diabetic retinopathy or hyperbaric oxygen-induced retinopathy, and conditions characterized by elevated intraocular pressure or secretion of ocular aqueous humor, such as glaucoma.

In one embodiment, the proliferative disease treated by the combination of the present invention is a cancer that can be beneficially treated by the inhibition of HSP90 and/or ALK including, for example, gastric, lung and bronchus; prostate; breast; pancreas; colon; rectum; thyroid; liver and intrahepatic bile duct; kidney and renal pelvis; urinary bladder; uterine corpus; uterine cervix; ovary; multiple myeloma; esophagus; acute myelogenous leukemia; chronic myelogenous leukemia; lymphocytic leukemia; myeloid leukemia; brain; oral cavity and pharynx; larynx; small intestine; non-Hodgkin lymphoma; melanoma; and villous colon adenoma.

In one embodiment, the proliferative disease treated by the combination of the present invention is a cancer of the esophagus, gastrointestinal cancer or gastric.

Where a tumor, a tumor disease, sarcoma, a carcinoma or a cancer are mentioned, also metastasis in the original organ or tissue and/or in any other location are implied alternatively or in addition, whatever the location of the tumor and/or metastasis.

The combination of the present invention is particularly useful for the treatment of proliferative diseases, particularly cancers and other malignancies, mediated by anaplastic lymphoma kinase (ALK). Proliferative diseases may include those showing overexpression or amplification of ALK, including lymphoma, osteosarcoma, melanoma, or a tumor of breast, renal, prostate, colorectal, thyroid, ovarian, pancreatic, neuronal, lung (non-small cell lung cancer and small cell lung cancer), uterine or gastrointestinal tumor, cancer of the bowel (colon and rectum), stomach cancer, cancer of liver, melanoma, bladder tumor, and cancer of head and neck. Hematological and neoplastic diseases, for example in anaplastic large-cell lymphoma (ALCL) and non-Hodgkin's lymphomas (NHL), specifically in ALK+NHL or Alkomas in inflammatory myofibroblastic tumors (IMT) and neuroblastomas.

In one embodiment, the present invention relates to a method for treating a proliferative disorder comprising administering to said subject a therapeutically effective amount of a compound of formula (I) and at least one Hsp90 inhibitor selected from the geldanamycin derivative, Tanespimycin (17-allylamino-17-demethoxygeldanamycin) (also known as KOS-953 and 17-AAG); Radicicol; 6-Chloro-9-(4-methoxy-3,5-dimethylpyridin-2-ylmethyl)-9H-purin-2-amine methanesulfonate (also known as CNF2024); IPI504; SNX5422; 5-(2,4-Dihydroxy-5-isopropyl-phenyl)-4-(4-morpholin-4-ylmethyl-phenyl)-isoxazole-3-carboxylic acid ethylamide (AUY922); and (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990) or a pharmaceutically acceptable salt thereof.

The present invention further relates to a kit comprising a compound of formula (I), or a pharmaceutically acceptable salt thereof, and at least one Hsp90 inhibitor or a pharmaceutically acceptable salt thereof, and a package insert or other labeling including directions for treating a proliferative disease.

The present invention further relates to a kit comprising a compound of formula (I), or a pharmaceutically acceptable salt thereof, and a package insert or other labeling including directions for treating a proliferative disease by co-administering at least one Hsp90 inhibitor or a pharmaceutically acceptable salt thereof.

Following is a description by way of example only.

### Example 1

### Antitumor effect of 5-{2,4-Dihydroxy-5-isopropyl-phersyS)-4-{4-morpholin-4-ylmethyl-phenyl)˜isoxazole-3-carboxylic acid ethylamide (AUY922) and 5-chloro-N2-(2-isopropoxy-5-methyl-4-(piperidin-4-yl)phenyl)-N4-[2-(propane-2-sulfonyl)-phenyl]-pyrimidine-2,4-diamine (Compound A) in the human lung primary tumor xenograft model HLUX1787

The subcutaneous human lung primary tumor xenograft model HLUX1787 harbors an EML4-ALK variant 2 translocation and has high levels of phospho-cMET. The primary tumor sample HLUX-1787 is a human primary tumor xenograft that is obtained from Oncology Research at Novartis Institute for Biomedical Research at Cambridge, Mass. The xenograft model was established by direct subcutaneous (sc) implantation of minced surgical material into the subcutaneous area of nude adult female mice. The tumors were then serially passaged in mice to enable studies in this report. HLUX-1787 primary tumors were harvested and cut into 3×3×3 mm³ size and implanted into nude mice. The tumors reached approximately 200 mm³ at 24-27 days post implantation. On Day 24 (TRP-0318) or Day 27 (TRP-0335), tumors were measured and mice were randomized into treatment groups based on tumor volume.

Compound A was dissolved in 0.5% MC/0.5% Tween 80. It is stable for at least one week at room temperature. The dosing volume was 10 ml/kg.

AUY922 (mesylate salt) was dissolved in 5% Dextrose in water (D5W), and prepared fresh before dosing. It was administered at 60.5 mg/kg (equivalent to 50 mg/kg free base), iv, twice a week (2qw) or once a week (qw).

### Efficacy Study Design

The designs for study TRP0318 and TRP0335 are summarized in Tables 1-1 and 1-2. Treatment dose was body weight adjusted. Tumor dimensions and body weights were collected at the time of randomization and twice weekly thereafter for the study duration. The following data were provided after each day of data collection: incidence of mortality, individual and group average body weight, and individual and group average tumor volume.

TABLE 1-1
Dose and Schedule for Study TRP0318

| | | | Number |
|---|---|---|---|
| Treatment | Dose | Schedule | of mice |
| D5W | 5 ml/kg | 2qw iv | 4 |
| 0.5% MC/ | 10 ml/kg | qd po | |
| 0.5% Tween 80 | | | |
| Compound A | 10 mg/kg | qd, po | 4 |
| AUY922 | 50 mg/kg | 2qw, iv | 4 |
| Compound A | 10 mg/kg | qd, po | 4 |
| AUY922 | 50 mg/kg | 2qw, iv | |

For study TRP0318, treatments were initiated on day 27 following tumor fragment implantation, when the average tumor volume was 240 mm³. Treatments continued for 20 days.

TABLE 1-2
Dose and Schedule for Study TRP0335

| | | | Number of |
|---|---|---|---|
| Treatment | Dose | Schedule | mice |
| D5W | 5 ml/kg | 2qw iv | 5 |
| 0.5% MC/ | 10 ml/kg | qd po | |
| 0.5% Tween 80 | | | |
| Compound A | 25 mg/kg | qd, po | 5 |
| AUY922 | 50 mg/kg | qw, iv | 5 |
| AUY922 | 50 mg/kg | 2qw, iv | 5 |
| Compound A | 25 mg/kg | qd, po | 5 |
| AUY922 | 50 mg/kg | qw, iv | |
| Compound A | 25 mg/kg | qd, po | 5 |
| AUY922 | 50 mg/kg | 2qw, iv | |

For study TRP0335, treatments were initiated on day 24 following tumor fragment implantation, when the average tumor volume was 240 mm³. Treatments continued for 13 days.

### Data Analysis

Body Weight

The % change in body weight was calculated as (BW_{current}−BWᵢₙᵢₜᵢₐₗ)/(BWᵢₙᵢₜᵢₐₗ)×100%. Data is presented as percent body weight change from the day of treatment initiation.

Tumor Volume

Percent treatment/control (T/C) values were calculated using the following formula:

% T/C=100×ΔT/ΔC if ΔT>0

% Regression=100×ΔT/Tᵢₙᵢₜᵢₐₗ if ΔT<0

where:

T=mean tumor volume of the drug-treated group on the final day of the study;

ΔT=mean tumor volume of the drug-treated group on the final day of the study—mean tumor volume of the drug-treated group on initial day of dosing;

Tᵢₙᵢₜᵢₐₗ=mean tumor volume of the drug-treated group on initial day of dosing;

C=mean tumor volume of the control group on the final day of the study; and

ΔC=mean tumor volume of the control group on the final day of the study—mean tumor volume of the control group on initial day of dosing.

Statistical Analysis

Tumor volume and percent body weight change were expressed as mean±standard error of the mean (SEM). Plasma concentration of compound was expressed as mean±standard deviation. Delta tumor volume was used for statistical analysis. Between group comparisons were carried out using the one way analysis of variance (ANOVA) followed by a post hoc Tukey test. For all statistical evaluations, the level of significance was set at p<0.05. Significance compared to the vehicle control group is reported unless otherwise stated.

### Results

Tolerability

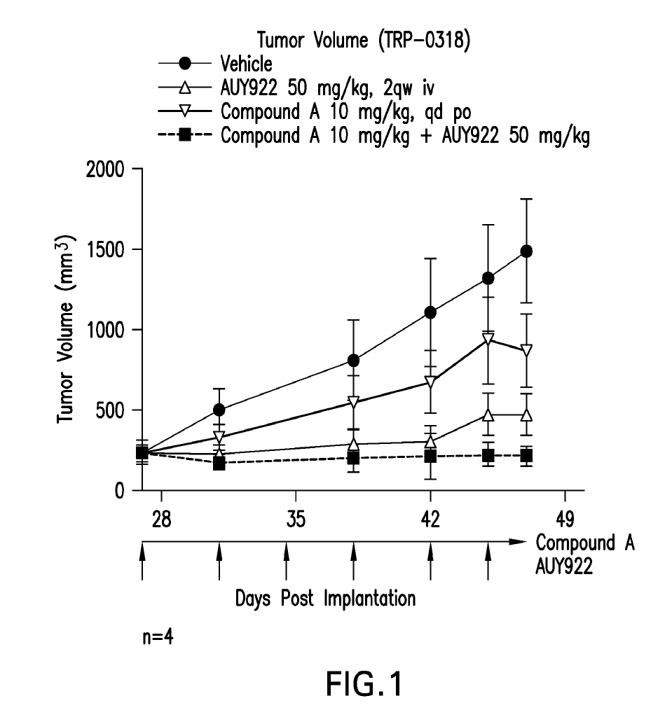

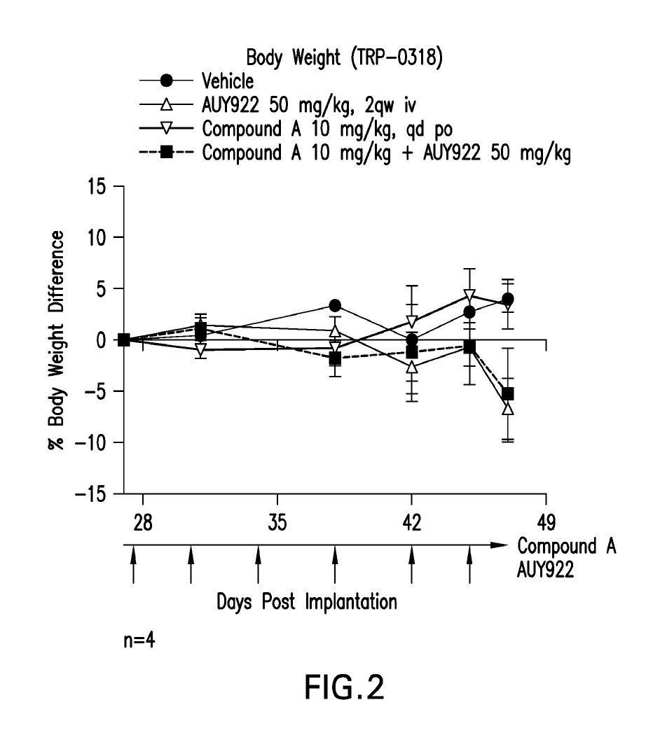

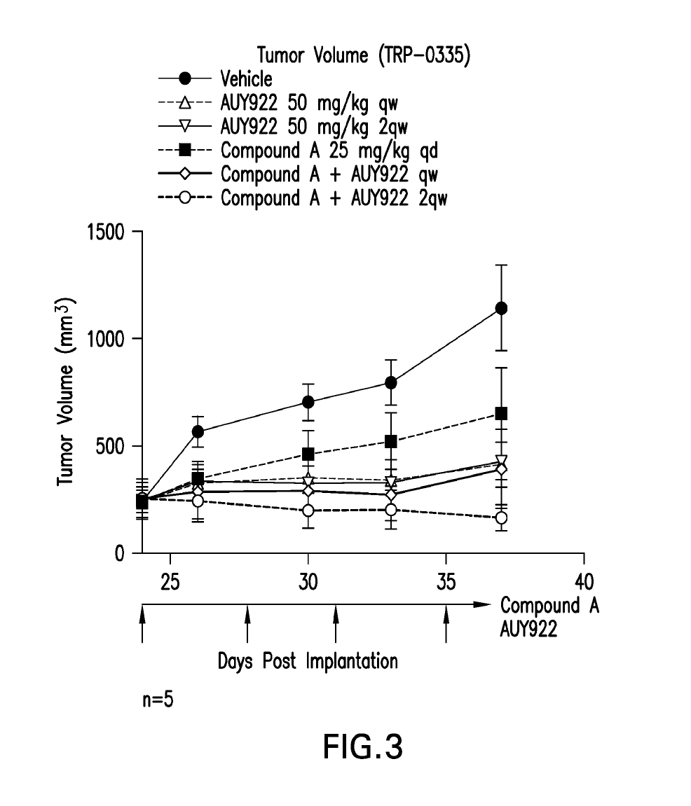

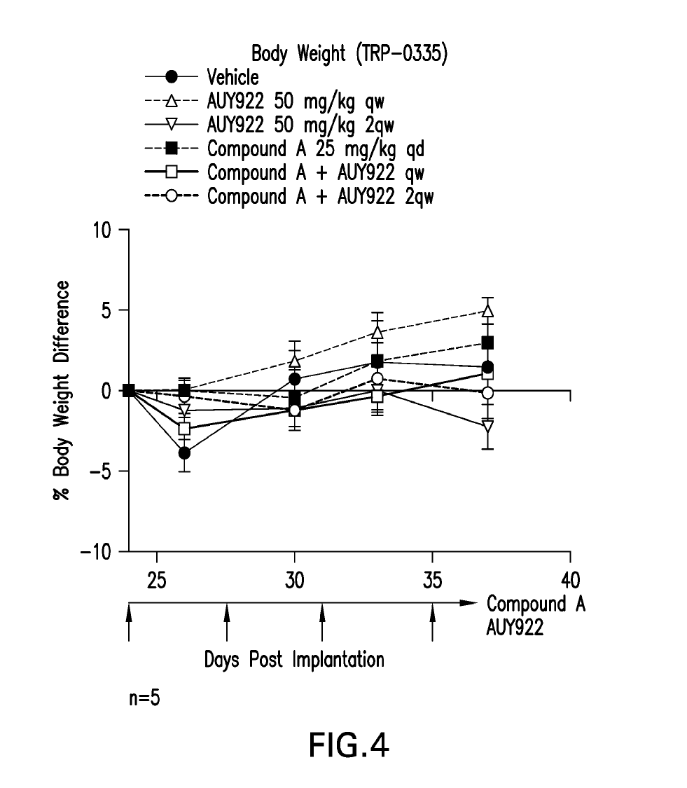

The initial mean body weight and percentage of body weight change at termination are summarized in Table 1-3 and shown in FIGS. 1 and 2 (TRP-0318), and summarized in Table 1-4 (TRP-0335) and shown in FIGS. 3 and 4.

TABLE 1-3
Mean initial body weight and percentage of
body weight change (TRP-0318)

| | | | % BW changes |
|---|---|---|---|
| Treatment | Dose/schedule | Initial BW (g) | on day 47 |
| D5W | 5 ml/kg, 2qw iv | 25.8 ± 0.7 | 4.1 ± 1.3 |
| 0.5% | 10 ml/kg, qd po | | |
| MC/0.5% | | | |
| Tween 80 | | | |
| Compound A | 10 mg/kg, qd po | 26.0 ± 0.3 | 3.5 ± 2.4 |
| AUY922 | 50 mg/kg, 2qw iv | 24.9 ± 0.5 | −6.8 ± 3.1 |
| AUY922 | 50 mg/kg, 2qw iv | 25.1 ± 0.7 | −5.2 ± 4.5 |
| Compound A | 10 mg/kg, qd po | | |

TABLE 1-4
Mean initial body weight and percentage of
body weight change (TRP-0335)

| | | | % BW changes |
|---|---|---|---|
| Treatment | Dose/schedule | Initial BW (g) | on day 37 |
| D5W | 5 ml/kg, 2qw iv | 25.2 ± 0.6 | 1.5 ± 3.2 |
| 0.5% | 10 ml/kg, qd po | | |
| MC/0.5% | | | |
| Tween 80 | | | |
| Compound A | 25 mg/kg, qd po | 25.1 ± 0.2 | 3.0 ± 2.2 |
| AUY922 | 50 mg/kg, qw iv | 24.2 ± 0.4 | 5.0 ± 0.8 |
| AUY922 | 50 mg/kg, 2qw iv | 24.6 ± 0.6 | −2.2 ± 1.4 |
| AUY922 | 50 mg/kg, qw iv | 25.3 ± 0.7 | 1.1 ± 0.7 |
| Compound A | 25 mg/kg, qd po | | |
| AUY922 | 50 mg/kg, 2qw iv | 26.0 ± 0.3 | −0.1 ± 1.6 |
| Compound A | 25 mg/kg, qd po | | |

In TRP-0318, Compound A was well tolerated at 10 mg/kg, with percent body weight change as 3.5%. The percent body weight change for the vehicle-treated group was 4.1% and the AUY922 50 mg/kg treated group was −6.8%. Compound A at 10 mg/kg in combination of AUY922 at 50 mg/kg twice a week resulted in −5.2% body weight losses.

Similarly, in TRP-0335, Compound A was well tolerated at 25 mg/kg with 3.0% body weight change, compared to vehicle-treated group with 1.5% body weight change, and AUY922 50 mg/kg once a week and twice a week treated group exhibit 5.0% and −2.2% body weight changes respectively. Compound A at 25 mg/kg in combination with AUY922 at 50 mg/kg once a week or AUY922 at 50 mg/kg twice a week, were also tolerated well with mean body weight change at 1.1% and −0.1% respectively.

In Vivo Efficacy

Tumor growth and percent T/C are summarized in Table 1-5 (TRP-0318) and Table 1-6 (TRP-0335) and illustrated in FIGS. 1 and 2 (TRP-0318) to FIGS. 3 and 4 (TRP-0335).

TABLE 1-5
Mean anti-tumor effect and body weight change summary
on day 47 (TRP-0318)

| | | | Tumor | | Host | |
|---|---|---|---|---|---|---|
| | | | Response | | Response | |
| | | | T/C | T/T0 | % BW | |
| Treatment | Dose | Schedule | (%) | (%) | change | Survival |
| D5W | 5 ml/kg | 2qw iv | | | 4.1% | 4 |
| 0.5% | 10 ml/kg | qd po | | | | |
| MC/0.5% | | | | | | |
| Tween 80 | | | | | | |
| Com- | 10 mg/kg | qd, po | 50.9% | | 3.5% | 4 |
| pound A | | | | | | |
| AUY922 | 50 mg/kg | 2qw, iv | 19.2%* | | −6.8% | 4 |
| Com- | 10 mg/kg | qd, po | | −6.8%* | −5.2% | 4 |
| pound A | | | | | | |
| AUY922 | 50 mg/kg | 2qw, iv | | | | |

*p < 0.05 compared to Vehicle by one way ANOVA post hoc Tukey test.

TABLE 1-6
Mean anti-tumor effect and body weight change summary
on day 37 (TRP-0335)

| | | | Tumor | | Host | |
|---|---|---|---|---|---|---|
| | | | Response | | Response | |
| | | | T/C | T/T0 | % BW | |
| Treatment | Dose | Schedule | (%) | (%) | change | Survival |
| D5W | 5 ml/kg | 2qw iv | | | 1.5% | 5 |
| 0.5% | 10 ml/kg | qd po | | | | |
| MC/0.5% | | | | | | |
| Tween 80 | | | | | | |
| Com- | 25 mg/kg | qd, po | 45.3% | | 3.0% | 5 |
| pound A | | | | | | |
| AUY922 | 50 mg/kg | qw, iv | 19.3%* | | 5.0% | 5 |
| AUY922 | 50 mg/kg | 2qw, iv | 20.0%* | | −2.2% | 5 |
| Com- | 25 mg/kg | qd, po | | | 1.1% | 5 |
| pound A | | | | | | |
| AUY922 | 50 mg/kg | qw, iv | 16.0%* | | | |
| Com- | 25 mg/kg | qd, po | | −34%** | −0.1% | 5 |
| pound A | | | | | | |
| AUY922 | 50 mg/kg | 2qw, iv | | | | |

*p < 0.05 compared to Vehicle by one way ANOVA post hoc Tukey test.
**p < 0.001 compared to Vehicle by one way ANOVA post hoc Tukey test.

In TRP-0318, Compound A at 10 mg/kg produced statistically non-significant anti-tumor effects with T/C 50.9%. AUY922 at 50 mg/kg resulted in T/C 19.2% (p<0.05 vs vehicle treated group), Compound A at 10 mg/kg in combination of AUY922 at 50 mg/kg twice a week resulted in tumor stasis with T/T0 −6.8% (p<0.05 vs vehicle treated group) (See Table 1-5, FIG. 1).

In TRP-0335, Compound A at 25 mg/kg resulted in statistically non-significant effects with T/C 45.3%. AUY922 at 50 mg/kg once a week and twice a week resulted in T/C 19.3% and 20.0%, respectively (p<0.05 vs vehicle treated group). Compound A at 25 mg/kg in combination of AUY922 at 50 mg/kg once a week resulted in T/C 16.0% (p<0.05 vs vehicle treated group); Compound A at 25 mg/kg in combination of AUY922 at 50 mg/kg twice a week resulted in tumor regression with T/T0 −34% (p<0.001 vs vehicle-treated group) (See Table 1-6, FIG. 3).

### Results

In the HLUX1787 model, Compound A at 10 mg/kg and 25 mg/kg yielded 50.9% T/C and 45.3% T/C respectively; AUY922 at 50 mg/kg (free base) twice weekly resulted in 20% T/C; combinations of Compound A at 10 mg/kg or 25 mg/kg with AUY922 at 50 mg/kg resulted in tumor stasis (T/T0: −6.8%) and tumor regression (T/T0: −34%) respectively. Increased antitumor effect was observed in the HLUX-1787 model when Compound A and the HSP90 inhibitor AUY922 were combined. The combination of Compound A with AUY922 is more potent than either single agent in a lung cancer model which harbors EML4-ALK variant 2 translocation.

### Example 2

### Antitumor effect of 5-{2,4-Dihydroxy-5-isopropyl-phersyS)-4-{4-morpholin-4-ylmethyl-phenyl)˜isoxazole-3-carboxylic acid ethylamide (AUY922) and 5-chloro-N2-(2-isopropoxy-5-methyl-4-(piperidin-4-yl)phenyl)-N4-[2-(propane-2-sulfonyl)-phenyl]-pyrimidine-2,4-diamine (Compound A) in the human lung primary tumor xenograft model LUF1656

The subcutaneous human lung primary tumor xenograft model LUF1656 harbors an EML4-ALK variant 1 translocation and has high levels of EGFR expression. EGFR, cMET and other RTK signaling pathways are also likely to be activated in these models.

### Experimental Design

TABLE 2-1
Dose and Schedule

| | | Compound 1 | | Compound 2 | | |
|---|---|---|---|---|---|---|
| | Number | | Dose and | | Dose and | |
| Group | of mice* | Drug | schedule | Drug | schedule | Endpoints |
| 1 | 8 | Vehicle 1 | 10 ml/kg po | Vehicle 2 | 5 ml/kg iv | Among the 8 mice in |
| | | (0.5% MC/0.5% | qd × 21 days | (D5W) | 2qwx 3 wks | each group, 4 mice |
| | | Tween 80) | | | | had tumor samples |
| 2 | 8 | Compound A | 25 mg/kg po | | | taken at 4 hrs after |
| | | | qd × 21 days | | | the last dose of |
| 3 | 8 | Compound A | 50 mg/kg po | | | Compound A or |
| | | | qd × 21 days | | | Vehicle 1. The rest |
| 4 | 8 | Compound A | 100 mg/kg po | | | of mice in each group |
| | | | qd × 21 days | | | were kept under |
| 5 | 8 | | | AUY922 | 50 mg/kg iv | observation for |
| | | | | | 2qw × 3 wks | 2 weeks. |
| 6 | 8 | Compound A | 25 mg/kg po | AUY922 | 50 mg/kg iv | |
| | | | qd × 21 days | | 2qw × 3 wks | |

### Methods

Tumor Inoculation

Tumor fragments from stock mice inoculated with selected primary human lung cancer (LUF1656) were harvested and used for inoculation into nu/nu mice. Each mouse was inoculated subcutaneously at the right flank with one tumor fragment (3×3×3 mm³) for tumor development. The treatments were started when mean tumor size reached approximately 140 mm³ (range 86.8-245 mm³). The test articles administration and the animal numbers in each group are shown in the experiment design Table 2-1.

TABLE 2-2
Testing Article Formulation Preparation

| | Dose | | Concentration | |
|---|---|---|---|---|
| Compounds | (mg/kg) | Preparation | (mg/ml) | Storage |
| Vehicle 1 for | — | 0.5% MC/0.5% Tween 80 | — | Stored at |
| Compound A | | | | 4° C. |
| Vehicle 2 for | | D5W | — | Stored at |
| AUY922 | | | | RT |
| Compound A | 100 | Suspended 370 mg Compound A in 37 ml | 10 | Stored at |
| (1) | | 0.5% methylcellulose/0.5% Tween 80, | | RT for |
| | | vortexed to mix well. | | 1 week |
| Compound A | 50 | Diluted 18 ml Compound A (1) in 18 ml 0.5% | 5 | Stored at |
| (2) | | methylcellulose/0.5% Tween 80. | | RT for |
| | | | | 1 week |
| Compound A | 25 | Diluted 17.5 ml Compound A (2) in 17.5 ml | 2.5 | Stored at |
| (3) | | 0.5% methylcellulose/0.5% Tween 80. | | RT for |
| | | | | 1 week |
| AUY922 | 50 | Dissolved 33.9 mg AUY922-AG (equivalent | 10 | Prepared |
| | | to 28 mg AUY922-NX) in 2.8 ml of D5W, | | fresh |
| | | sonicated until clear. | | |

Tumor Measurements and the Endpoints

The major endpoint was to see if the tumor growth can be delayed or tumor bearing mice can be cured. Tumor size was measured twice weekly in two dimensions using a caliper, and the volume was expressed in mm³ using the formula: V=0.5a×b² where a and b are the long and short diameters of the tumor, respectively. The tumor size was then used for calculations of both T-C and T/C values. T-C was calculated with T as the time (in days) required for the mean tumor size of the treatment group to reach a predetermined size (e.g., 400 mm³), and C was the time (in days) for the mean tumor size of the control group to reach the same size. Percent treatment/control (T/C) values were calculated using the following formula:

% T/C=100×ΔT/ΔC if ΔT>0%

% Regression=100×ΔT/Mᵢₙᵢₜᵢₐₗ if ΔT<0

where:

T=mean tumor volume of the drug-treated group on the final day of the study;

ΔT=mean tumor volume of the drug-treated group on the final day of the study—mean tumor volume of the drug-treated group on initial day of dosing;

Tᵢₙᵢₜᵢₐᵢ=mean tumor volume of the drug-treated group on initial day of dosing;

C=mean tumor volume of the control group on the final day of the study; and

ΔC=mean tumor volume of the control group on the final day of the study—mean tumor volume of the control group on initial day of dosing.

Statistical Analysis

Summary statistics, including mean and the standard error of the mean (SEM), are provided for the tumor volume of each group at each time point.

Statistical analysis of difference in tumor volume among the groups was conducted using a one-way ANOVA followed by multiple comparisons using Tukey HSD. Log transformation was performed for homogeneity of variances when necessary. All data were analyzed using SPSS (Statistical Package for the Social Sciences or Statistical Product and Service Solutions) 16.0. p <0.05 was considered to be statistically significant.

The standard protocols used in pharmacology studies are not pre-powered to demonstrate statistically significant superiority of a combination over the respective single agent treatment. The statistical power is often limited by potent single agent response and/or model variability. The p-values for combination vs single agent treatments are, however, provided.

### Results

Body Weights

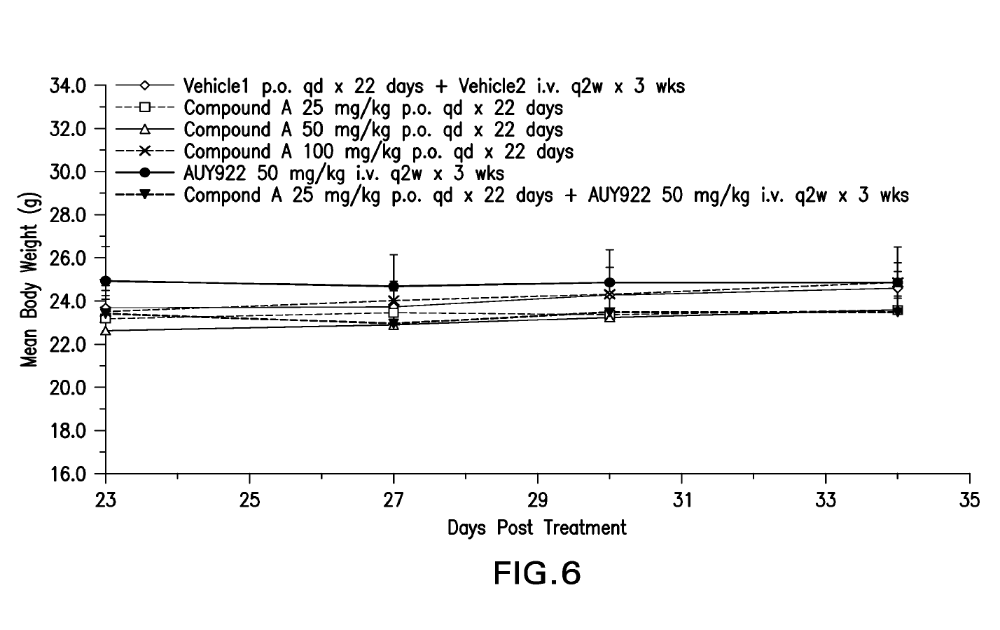

The results of the body weight changes in the tumor bearing mice are shown in FIG. 5 and FIG. 6.

### Tumor Volumes

The tumor sizes of the different groups at different time points are shown in Table 2-3 and Table 2-4.

TABLE 2-3
Tumor Sizes in the Different Treatment Groups (treatment phase, n = 8)

| | Tumor Volume (mm³)^{a} | | | | | |
|---|---|---|---|---|---|---|
| | | | | | | Cmpd A |
| | | | | | | 25 mg/kg (QD × |
| | | Cmpd A | | | AUY922 | 22 Days) |
| Days | | 25 mg/kg | Cmpd A | Cmpd A | 50 mg/kg | AUY922 |
| post | Vehicle 1 + | (QD × | 50 mg/kg (QD × | 100 mg/kg | (2qw × | 50 mg/kg |
| Treatment | Vehicle 2 | 22 Days) | 22 Days) | (QD × 22 Days) | 3 wks) | (2qw × 3 wks) |
| 0 | 139.5 ± 17.0 | 139.8 ± 16.7 | 139.5 ± 17.0 | 139.4 ± 18.3 | 140.1 ± 17.3 | 139.5 ± 15.6 |
| 4 | 226.7 ± 45.2 | 171.5 ± 29.9 | 144.9 ± 23.5 | 110.8 ± 21.7 | 177.5 ± 22.9 | 112.7 ± 20.7 |
| 7 | 283.7 ± 54.6 | 205.4 ± 46.4 | 138.8 ± 30.8 | 107.7 ± 24.6* | 194.9 ± 28.0 | 112.5 ± 26.5* |
| 11 | 416.0 ± 78.5 | 248.0 ± 68.4 | 155.4 ± 38.8** | 118.3 ± 29.9** | 244.5 ± 32.2 | 121.2 ± 34.6** |
| 14 | 552.0 ± 103.3 | 296.9 ± 93.9 | 175.1 ± 45.2** | 133.2 ± 33.2** | 282.1 ± 36.7 | 147.3 ± 48.4** |
| 18 | 750.0 ± 141.1 | 356.1 ± 113.6 | 194.5 ± 53.6** | 146.1 ± 36.4*** | 402.5 ± 51.9 | 209.5 ± 72.9** |
| 21 | 983.2 ± 198.1 | 435.7 ± 155.6 | 231.5 ± 65.2** | 155.8 ± 41.2*** | 466.8 ± 59.5 | 235.7 ± 86.8** |

Note:
^{a}Mean ± SEM;
n: animal number;
*P < 0.05, **P < 0.01, ***P < 0.001, compared with the vehicle control.

TABLE 2-4
Tumor Sizes in the Different Treatment Groups (re-growth phase, n = 4)

| | Tumor Volume (mm³) | | | | | |
|---|---|---|---|---|---|---|
| | | Compound | Compound | Compound | | Compound A |
| | | A | A | A | | 25 mg/kg (QD × |
| Days | | 25 mg/kg | 50 mg/kg | 100 mg/kg | AUY922 50 mg/kg | 22 Days) |
| post | Vehicle 1 + | (QD × | (QD × | (QD × | (2qw × | AUY922 50 mg/kg |
| Treatment | Vehicle 2 | 22 Days) | 22 Days) | 22 Days) | 3 wks) | (2qw × 3 wks) |
| 23 | 1085.3 ± 310.8 | 434.4 ± 141.0 | 270.1 ± 109.0 | 186.4 ± 68.1 | 612.0 ± 80.7 | 254.6 ± 94.4 |
| 27 | 1324.6 ± 378.7 | 552.4 ± 159.3 | 300.2 ± 106.2 | 203.2 ± 77.3 | 904.7 ± 136.8 | 352.5 ± 126.0 |
| 30 | 1574.8 ± 432.7 | 671.6 ± 175.7 | 348.5 ± 124.4 | 235.0 ± 93.9 | 1136.2 ± 188.6 | 497.6 ± 173.6 |
| 34 | 1924.3 ± 499.2 | 949.9 ± 246.7 | 514.3 ± 163.8 | 304.0 ± 120.3 | 1508.9 ± 273.8 | 766.9 ± 275.5 |

### Tumor Growth Inhibition

The tumor growth inhibition is summarized in Table 2-5.

TABLE 2-5
Antitumor Activity of Compound A as a Single Agent and in
Combination with AUY922 in the Treatment of Primary Human
Lung Cancer LUF1656 Xenograft Model at Day 21.

| | Tumor Size | | |
|---|---|---|---|
| | (mm³)^{a} at | | |
| | Day 21 after | | P |
| Treatment | Treatment | T/C (%) | value^{b} |
| Vehicle 1 + Vehicle 2 | 983.2 ± 198.1 | — | — |
| Compound A | 435.7 ± 155.6 | 35.1 | 0.098 |
| (25 mg/kg, PO, QD × 22 Days) | | | |
| Compound A | 231.5 ± 65.2 | 10.9 | 0.002 |
| (50 mg/kg, PO, QD × 22 Days) | | | |
| Compound A | 155.8 ± 41.2 | 1.9 | <0.001 |
| (100 mg/kg, PO, QD × 22 Days) | | | |
| AUY922 | 466.8 ± 59.5 | 38.7 | 0.486 |
| (50 mg/kg, IV, 2QW × 3 wks) | | | |
| Compound A | 235.7 ± 86.8 | 11.4 | 0.001 |
| (25 mg/kg, PO, QD × 22 Days) + | | | |
| AUY922 | | | |
| (50 mg/kg, IV, 2QW × 3 wks) | | | |

Note:
^{a}Mean ± SEM;
^{b}vs. vehicle control.

### Tumor Growth Curves

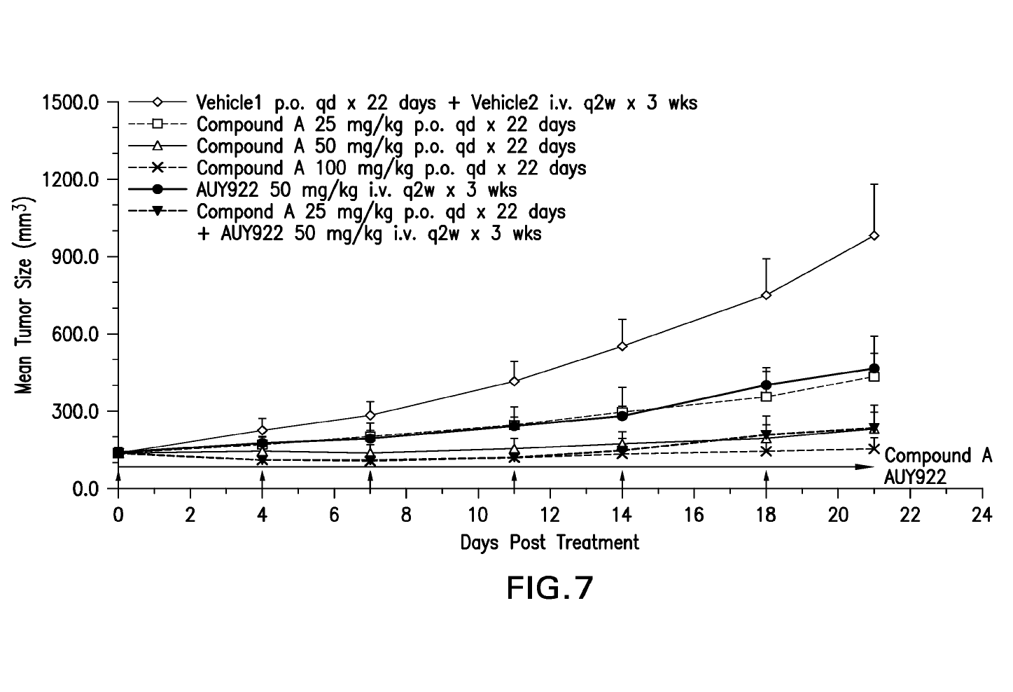

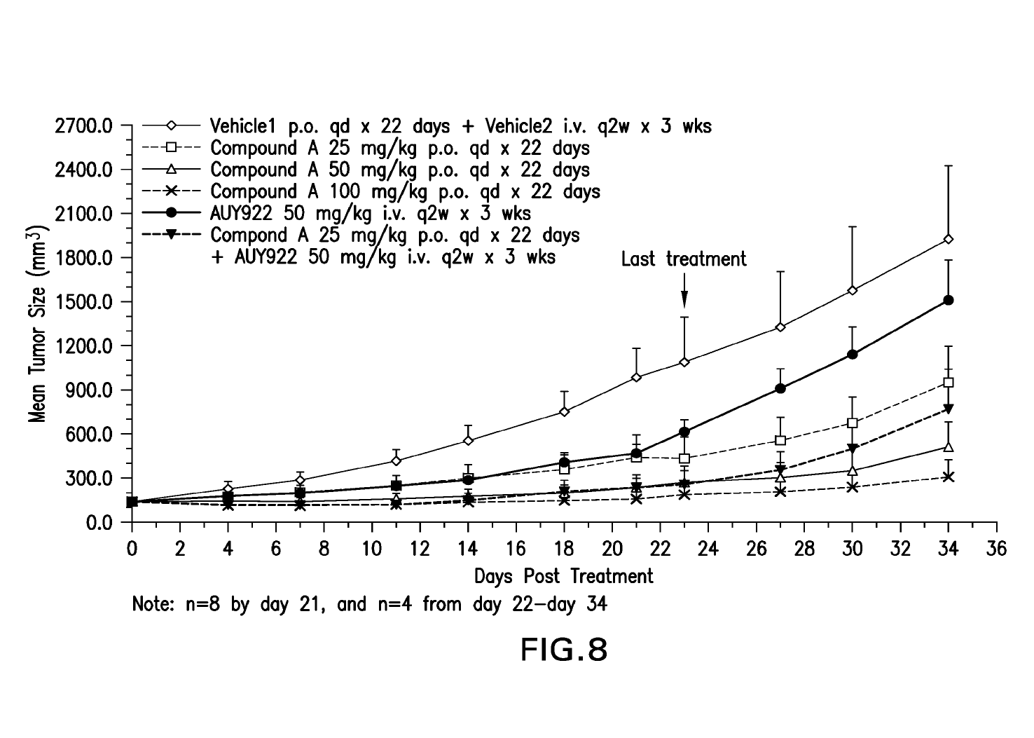

The tumor growth curves of different groups are shown in FIGS. 7 and 8.

### Result Summary and Discussion

In this efficacy study, the therapeutic efficacy of Compound A as a single agent and in combination with AUY922 in the treatment of subcutaneous primary human lung cancer LUF1656 xenograft model in nu/nu mice was evaluated. The results of tumor size in different groups at different time points after treatment are shown in the Tables 2-3 and 2-4 and in FIGS. 7 and 8.

Treatment with Compound A as a single agent at 25 mg/kg (PO, QD×22 Days) showed moderate antitumor activity (T/C value=35.1% on Day 21 after treatment) (p>0.05 when compared to vehicle). Treatment with Compound A as a single agent at 50 and 100 mg/kg (PO, QD×22 Days) exhibited significant antitumor activity from Day 11 to Day 21 and Day 7 to Day 21 after treatment compared with vehicle control (T/C value=10.9%, p<0.01, at Day 21 after treatment of 50 mg/kg Compound A treatment group; and T/C value=1.9%, p<0.001, at Day 21 after treatment of 100 mg/kg Compound A treatment group). Treatment with AUY922 as a single agent at 50 mg/kg (IV, 2QW×3 wks) showed moderate antitumor activity (T/C value=38.7% at Day 21 after treatment when compared to vehicle). Treatment with 25 mg/kg Compound A (PO, QD×22 Days) plus 50 mg/kg AUY922 (IV, 2QW×3 wks) showed significant antitumor activity from Day 7 to Day 21 after treatment when compared to vehicle control (T/C value=11.4%, p<0.01, at Day 21 after treatment). The antitumor activity of the combination treatment (25 mg/kg Compound A+50 mg/kg AUY922) was better than that of each monotherapy.

Based on the body weight data as shown in FIGS. 5 and 6, the test articles Compound A at dose levels of 25, 50 and 100 mg/kg, AUY922 at 50 mg/kg and combination of 25 mg/kg Compound A with 50 mg/kg AUY922 were all tolerated by the primary human lung cancer LUF1656 tumor-bearing mice in this study.

In summary, the test article Compound A at 50 and 100 mg/kg as single agent and 25 mg/kg Compound A in combination with 50 mg/kg AUY922 all demonstrated statistically significant antitumor activity against the primary human lung cancer LUF1656 xenograft model. Combination of Compound A and AUY922 produced increased anti-tumor activity compared to the corresponding monotherapies.

1. A pharmaceutical combination comprising;
(a) compound having Formula (1): 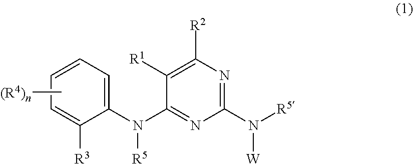
or pharmaceutically acceptable salts thereof; wherein 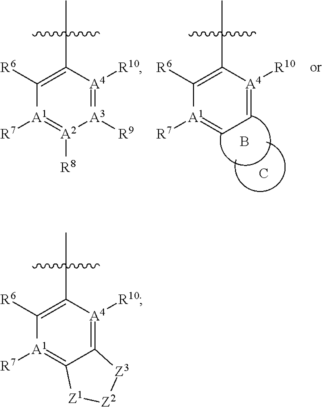
W is
A¹ and A⁴ are independently C or N;
each A² and A³ is C, or one of A² and A³ is N when R⁸ and R⁷ form a ring;
B and C are independently an optionally substituted 5-7 membered carbocyclic ring, aryl, heteroaryl or heterocyclic ring containing N, O or S;
Z¹, Z² and Z³ are independently NR¹¹, C═O, CR—OR, (CR₂)₁₋₂ or ═C—R¹²;
R¹ and R² are independently halo, OR¹², NR(R¹²), SR¹², or an optionally substituted C₁₋₆ alkyl, C₂₋₆ alkenyl or C₂₋₆ alkynyl; or one of R¹ and R² is H;
R³ is (CR₂)₀₋₂SO₂R¹², (CR₂)₀₋₂SO₂NRR¹², (CR₂)₀₋₂CO₁₋₂R¹², (CR₂)₀₋₂CONRR¹² or cyano;
R⁴, R⁶, R⁷ and R¹⁰ are independently an optionally substituted C₁₋₆ alkyl, C₂₋₆ alkenyl or C₂₋₆ alkynyl; OR¹², NR(R¹²), halo, nitro, SO₂R¹², (CR₂)ₚR¹³ or X; or R⁴, R⁷ and R¹⁰ are independently H;
R, R⁵ and R^{5′} are independently H or C₁₋₆ alkyl;
R⁸ and R⁹ are independently C₁₋₆ alkyl, C₂₋₆ alkenyl, C₂₋₅ alkynyl, halo or X, or one of R⁸ and R⁹ is H when R¹ and R² form a ring; and provided one of R⁸ and R⁹ is X;
alternatively, R¹ and R², or R⁶ and R⁷, R⁷ and R⁸, or R⁹ and R¹⁰, when attached to a carbon atom may form an optionally substituted 5-7 membered monocyclic or fused carbocyclic ring, aryl, or heteroaryl or heterocyclic ring comprising N, O and/or S; or R⁷, R⁸, R⁹ and R¹⁰ are absent when attached to N;
R¹¹ is H, C₁₋₆ alkyl, C₂₋₆ alkenyl, (CR₂)ₚCO₁₋₂R, (CR₂)ₚOR, (CR₂)ₚR¹³, (CR₂)ₚNRR¹², (CR₂)ₚCONRR¹² or (CR₂)ₚSO₁₋₂R¹²;
R¹² and R¹³ are independently an optionally substituted 3-7 membered saturated or partially unsaturated carbocyclic ring, or a 5-7 membered heterocyclic ring comprising N, O and/or S; aryl or heteroaryl; or R¹² is H, C₁₋₆alkyl;
X is (CR₂)_{q}Y, cyano, CO₁₋₂R¹², CONR(R¹²), CONR(CR₂)ₚNR(R¹²), CONR(CR₂)ₚOR¹², CONR(CR₂)ₚSR¹², CONR(CR₂)ₚS(O)₁₋₂R¹² or (CR₂)₁₋₆NR(CR₂)ₚOR¹²;
Y is an optionally substituted 3-12 membered carbocyclic ring, a 5-12 membered aryl, or a 5-12 membered heteroaryl or heterocyclic ring comprising N, O and/or S and attached to A² or A³ or both via a carbon atom of said heteroaryl or heterocyclic ring when q in (CR₂)ₚY is 0; and
n, p and q are independently 0-4; and
(b) at least one Hsp90 inhibitor or a pharmaceutically acceptable salt thereof.

2. A combination according to claim 1 wherein said agent (a) is selected from 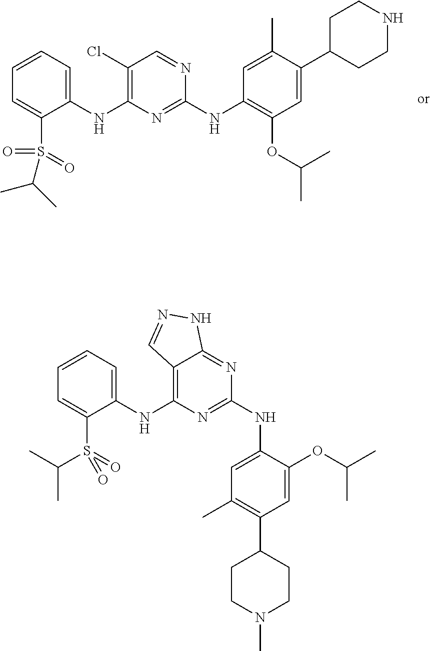 [SMILES: Cc1cc(Nc2ncc(Cl)c(Nc3ccccc3S(=O)(=O)C(C)C)n2)c(OC(C)C)cc1C1CCNCC1 ; Cc1cc(Nc2nc(Nc3ccccc3S(=O)(=O)C(C)C)c3cn[nH]c3n2)c(OC(C)C)cc1C1CCN(C)CC1]
or pharmaceutically acceptable salts thereof.

3. A pharmaceutical combination according to claim 1, wherein agent (b) is selected from the geldanamycin derivative, Tanespimycin (17-allylamino-17-demethoxygeldanamycin)(also known as KOS-953 and 17-AAG); Radicicol; 6-Chloro-9-(4-methoxy-3,5-dimethylpyridin-2-ylmethyl)-9H-purin-2-amine methanesulfonate (also known as CNF2024); IP1504; SNX5422; 5-(2,4-Dihydroxy-5-isopropyl-phenyl)-4-(4-morpholin-4-ylmethyl-phenyl)-isoxazole-3-carboxylic acid ethylamide (AUY922); and (R)-2-amino-7-[4-fluoro-2-(6-methyoxy-pyridin-2-yl)-phenyl]-4-methyl-7,8-dihydro-6H-pyrido[4,3-d]pyrimidin-5-one (HSP990) or a pharmaceutically acceptable salt thereof.

4. A pharmaceutical combination according to claim 3, wherein agent (b) is Dihydroxy-5-isopropyl-phenyl)-4-(4-morpholin-4-ylmethyl-phenyl)-isoxazole-3-carboxylic acid ethylamide (AUY922).

5. A pharmaceutical combination according to claim 1 for simultaneous, separate or sequential use for the treatment of a proliferative disease.

6. A pharmaceutical combination according to claim 5, wherein the proliferative disease is a lymphoma; anaplastic large-cell lymphoma; osteosarcoma; neuroblastoma; inflammatory myofibroblastic tumors tumor of lung and bronchus; prostate; breast; pancreas; colon; rectum; thyroid; liver and intrahepatic bile duct; kidney and renal pelvis; urinary bladder; uterine corpus; uterine cervix; ovary; myeloma; multiple myeloma; esophagus; acute myelogenous leukemia; chronic myelogenous leukemia; lymphocytic leukemia; myeloid leukemia; brain; oral cavity and pharynx; larynx; small intestine; stomach; gastrointestinal; head and neck; non-Hodgkin lymphoma; melanoma; or villous colon adenoma.

7. A pharmaceutical composition comprising a compound of formula I according to claim 1 or a pharmaceutically acceptable salt thereof and at least one Hsp90 inhibitor or a pharmaceutically acceptable salt thereof for use in the treatment of a proliferative disease.

8. (canceled)

9. (canceled)

10. A method for treating a proliferative disease in a subject in need thereof, comprising administering to said subject a therapeutically effective amount of a compound of formula (I) according to claim 1 or a pharmaceutically acceptable salt thereof, and at least one Hsp90 inhibitor or a pharmaceutically acceptable salt thereof.

11. A method for treating a proliferative disease according to claim 7, wherein said agent (a) is selected from 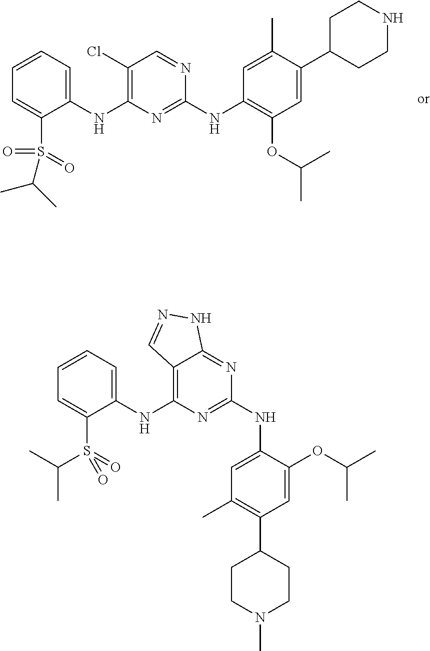 [SMILES: Cc1cc(Nc2ncc(Cl)c(Nc3ccccc3S(=O)(=O)C(C)C)n2)c(OC(C)C)cc1C1CCNCC1 ; Cc1cc(Nc2nc(Nc3ccccc3S(=O)(=O)C(C)C)c3cn[nH]c3n2)c(OC(C)C)cc1C1CCN(C)CC1]
or pharmaceutically acceptable salts thereof.

12. A method for treating a proliferative disease according to claim 7, wherein the Hsp90 inhibitor is selected from the geldanamycin derivative, Tanespimycin (17-allylamino-17-demethoxygeldanamycin)(also known as KOS-953 and 17-AAG); Radicicol; 6-Chloro-9-(4-methoxy-3,5-dimethylpyridin-2-ylmethyl)-9H-purin-2-amine methanesulfonate (also known as CNF2024); IP1504; SNX5422; 5-(2,4-Dihydroxy-5-isopropyl-phenyl)-4-(4-morpholin-4-ylmethyl-phenyl)-Isoxazole-3-carboxylic acid ethylamide (AUY922); and (R)-2-amino-7-[4-fluoro-2-(6-methyoxy-pyridin-2-yl)-phenyl}-4-methyl-7,8-dihydro-6H-pyrido[4,3-d]pyrimidin-5-one (HSP990).

13. A method according to claim 10, wherein the compound of formula (I) and the Hsp90 inhibitor are administered together as a single pharmaceutical composition.

14. A method according to claim 10, wherein the compound of formula (I) and the Hsp90 inhibitor are administered as separate compositions or sequentially.

15. A kit comprising a compound of formula (I) according to claim 1 or a pharmaceutically acceptable salt thereof, and a package insert or label providing directions for treating a proliferative disease by co-administering at least one Hsp90 inhibitor or a pharmaceutically acceptable salt thereof.

16. A method for treating a proliferative disease according to claim 10 wherein said proliferative disease is selected from lymphoma; anaplastic large-cell lymphoma; osteosarcoma; neuroblastoma; inflammatory myofibroblastic tumors tumor of lung and bronchus; prostate; breast; pancreas; colon; rectum; thyroid; liver and intrahepatic bile duct; kidney and renal pelvis; urinary bladder; uterine corpus; uterine cervix; ovary; myeloma; multiple myeloma; esophagus; acute myelogenous leukemia; chronic myelogenous leukemia; lymphocytic leukemia; myeloid leukemia; brain; oral cavity and pharynx; larynx; small intestine; stomach; gastrointestinal; head and neck; non-Hodgkin lymphoma; melanoma; or villous colon adenoma.
